(19) United States
(12) Patent Application Publication
(10) Pub. No.: US 2010/0004244 A1
(43) Pub. Date: Jan. 7, 2010

(54) USE OF CB2 RECEPTOR AGONISTS FOR PROMOTING NEUROGENESIS

(75) Inventors: Ismael Galve-Roperh, Madrid (ES); Manuel Guzman, San Bernardo (ES); Raphael Mechoulam, Jerusalem (ES); Javier Palazuelos, Madrid (ES); Tania Aguado, Madrid (ES)

Correspondence Address: WINSTON & STRAWN LLP;PATENT DEPARTMENT, 1700 K STREET, N.W., WASHINGTON, DC, 20006, US

(73) Assignee: YISSUM RESEARCH DEVELOPMENT COMPANY OF THE HEBREW UNIVERSITY OF JERUSALEM, Jerusalem (IL)

(21) Appl. No.: 12/306,636

(22) PCT Filed: Jun. 27, 2007
(86) PCT No.: PCT/IL07/00785

§ 371 Date: Aug. 11, 2009

Related U.S. Application Data
(60) Provisional application No. 60/816,591, filed on Jun. 27, 2006.

(51) Int. Cl. A61K 31/53; A61K 31/50; A61K 31/517; A61K 31/47; A61K 31/42; A61K 31/415; A61K 31/404; A61K 31/18; A61K 31/166; A61K 31/16

(52) U.S. Cl. 514/241; 514/252.01; 514/266.1; 514/311; 514/374; 514/406; 514/415; 514/601; 514/617; 514/627

(57) ABSTRACT

The present invention relates to ligands of the peripheral cannabinoid receptor CB₂, especially (+)-α-pinene derivatives, and to pharmaceutical compositions thereof, which are useful for promoting, inducing and enhancing neurogenesis including neural cell regeneration. In particular, pharmaceutical compositions of the invention will be useful for preventing, alleviating or treating neurological injuries or damages to the CNS or the PNS associated with physical injury, ischemia, neurodegenerative disorders, certain medical procedures or medications, tumors, infections, metabolic or nutritional disorders, cognition or mood disorders, and various medical conditions associated with neural damage or destruction.

### FIELD OF THE INVENTION

The present invention relates to ligands of the peripheral cannabinoid receptor CB₂, especially (+)-α-pinene derivatives, and to pharmaceutical compositions thereof, which are useful for promoting, inducing and enhancing neurogenesis.

### BACKGROUND OF THE INVENTION

Unlike cells in many tissues, which undergo generation and replacement throughout life, most neurons of the mammalian brain are entirely generated during early development and are not replaced if lost. The failure of the mammalian nervous system to regenerate after injury is a major clinical problem. Though endogenous molecules can promote axonal growth, at least three factors are responsible for lack of regeneration of nerve fibers: the formation of a glial scar, the presence of inhibitors of regeneration in myelin, and the intrinsic growth capacity of adult axons.

Damage to the nervous system, both central (CNS) and peripheral (PNS), can result from several causes including physical injury, ischemia, neurological disorders, certain medical procedures or therapies, tumors, infections, metabolic or nutritional disorders, cognition or mood disorders, and various diseases. Together these medical conditions occur with a high incidence among the population and result in a severe unmet medical need. In recent years, neurodegenerative disease has become an increasingly important concern due to the expanding elderly population. Neural damage as a result of stroke or trauma to the brain or spinal cord is also a leading cause of death and disability. Since the nervous system cannot undergo regeneration, damage in particular to the brain, spinal cord, and optic nerve is believed to be irreversible, leading ultimately to permanent impairment of motor and sensory functions.

Despite intensive research, there are currently no effective methods that can promote significant nerve regeneration of severed or damaged nerve fibers. The therapeutic approaches include promoting the activity of the endogenous molecules involved in the extension of axonal growth cones, blocking of the inhibitors of regeneration or altering the growth capacity of the neural cells or tissue.

Cannabis was historically used for the treatment of insomnia, inflammation, pain, various psychoses, digestive disorders, depression, migraine, fatigue, infections and appetite disorders. Originally defined as any individual bioactive component of the plant cannabis, the cannabinoids have come to encompass their endogenous counterparts and any synthetic compound that would exert most of its actions via the activation of the specific G-protein coupled cannabinoid receptors. To date, two cannabinoid receptors have been cloned and characterized, cannabinoid receptor type 1 (CB₁) and cannabinoid receptor type 2 (CB₂), although additional receptors may exist [Begg, M. et al., Pharmacology & Therapeutics 106, 133-145, 2005].

The CB₁ receptors, responsible among other things for the psychotropic effects of cannabinoids, are predominantly found in the central nervous system (CNS) where they are expressed by the major types of brain cells: neurons [Herkenham, M. et al., Proc. Natl. Acad. Sci. USA 87, 1932-6, 1990], astrocytes [Bouaboula, M. et al., J. Biol. Chem. 270, 13973-80, 1995], oligodendrocytes [Molina-Holgado, E. et al., J. Neurosci. 22, 9742-53, 2002] and microglia [Sinha, D. et al., J. Neuroimmunol. 82, 13-21, 1998]. Functionally active CB₁ receptors are also expressed in peripheral nerve terminals and various extra-neural sites such as testis, eye, vascular endothelium and spleen [Howlett, A. C. et al., Pharmacol. Rev. 54, 161-202, 2002; Piomelli, D., Nat. Rev. Neurosci. 4, 873-884, 2003].

The CB₂ receptor displays a more limited pattern of expression, being found almost exclusively in cells (e.g. B- and T-lymphocytes, macrophages) and tissues (e.g. spleen, tonsils, lymph nodes) of the immune system [Walter, L. and Stella, N., Br. J. Pharmacol. 141, 775-85, 2004]. Within the brain, the CB₂ receptor seems to be solely expressed in perivascular microglial cells [Nunez, E. et al., Synapse 53: 208-13, 2004], vascular endothelial cells [Golech, S. A. et al., Brain Res. Mol. Brain Res. 132, 87-92, 2004] and certain neuron subpopulations [Van Sickle, M. D. et al., Science 310, 329-32, 2005; Gong, J. et al., Brain Res. 1071, 10-23, 2006; Ashton, J. C., et al., Neurosci Lett. 396, 113-6, 2006]. This restricted expression pattern in the brain makes however the CB₂ receptor an interesting therapeutic target since the unwanted psychotropic effects of cannabinoids, which severely limit their medical use, are mediated largely or entirely by neuronal CB₁ receptors. While CB₂ receptor expression in the brain has been examined to date only in differentiated cells, the presence and function of this receptor in neural progenitor cells remain unknown.

To date the effect of cannabinoids on cellular growth was mainly reported for cells derived from malignancies and components of the immune system. These studies have generally suggested an inverse relation between CB₂ receptor expression and stage of cell differentiation [Guzman, M. et al., Pharmacol. Ther. 95, 175-84, 2002].

Only recently, studies have suggested that the hippocampus [Eriksson, P. S. et al., Nat. Med. 4, 1313-7, 1998], and possibly higher processing centers of the brain such as the neocortex [Gould, E. et al., Science 286, 548-52, 1999], are able to generate new neurons. The adult regenerated neurons are integrated into the existing brain circuitry, where they contribute to ameliorate neurological deficits.

The finding that CB₁ knockout mice suffered from prominently decreased hippocampal neurogenesis led to the hypothesis that ligand(s) to this predominantly brain expressed receptor could be involved in neurogenesis [Aguado, T. et al., FASEB J. 19, 1704-6, 2005]. After establishing that the CB₁ receptor is present in both embryonic and adult rat hippocampal neural stem cells, Jiang et al. tested the ability of the potent synthetic cannabinoid HU-210 to promote proliferation of embryonic hippocampal neural progenitor cells [Jiang, W. et al., Journal of Clinical Investigation 115, 3104-16, 2005]. They found that HU-210 promotes proliferation of said embryonic cells without affecting their differentiation. The effect of HU-210 on neural proliferation was blocked by a selective CB₁ antagonist. Chronic administration of HU-210 increased the number of newborn neurons and reduced measures of anxiety- and depression-like behavior. Jiang and co-workers did not attribute a role to the CB₂ receptor or agonists thereof.

The tissue distribution of the cannabinoid receptors and the psychoactive effects mediated by the CB₁ receptor have led the scientific and clinical community to prefer, whenever possible, CB₂ selective agonists. Such compounds were shown to have neuroprotective effects especially in neuroinflammatory diseases and in models of neurotoxic degeneration mimicking neurodegenerative disorders. However, neuroprotection based on blocking of degenerative processes does not teach that compounds may have neurogenic properties, which open opportunities for new therapeutic uses involving boosting of the brain restorative potential.

U.S. Pat. No. 4,282,248 discloses both isomeric mixtures and individual isomers of pinene derivatives. Therapeutic activity, including analgesic, central nervous system depressant, sedative and tranquilizing activity, was attributed to the compounds, but the disclosure does not teach that these compounds bind to any cannabinoid receptor.

U.S. Pat. No. 5,434,295 discloses a family of novel 4-phenyl pinene derivatives, and teaches how to utilize these compounds in pharmaceutical compositions useful in treating various pathological conditions associated with damage to the central nervous system. U.S. Pat. No. 5,434,295 neither teaches nor suggests that any of the disclosed compounds are selective for peripheral cannabinoid receptors and the physiological examples suggest that these compounds might act through blocking of the NMDA receptor. Though the neuroprotective activity of these compounds encompass the treatment of certain chronic degenerative diseases which are characterized by gradual selective neuronal loss through apoptosis or necrosis, neuroregenerative properties are not disclosed.

U.S. Pat. Nos. 6,864,291 and 6,903,137 disclose a family of bicyclic compounds, including (+){4-[4-(1,1-dimethylheptyl)-2,6-dimethoxy-phenyl]-6,6-dimethyl-bicyclo[3.1.1]hept-2-en-2-yl}-methanol (designated HU-308), as CB₂ specific agonists and exemplifies their use in the treatment of pain and inflammation, autoimmune diseases, gastrointestinal disorders and as hypotensive agents.

International (PCT) Patent application WO 03/064359 discloses that the CB₂ specific agonist HU-308 is useful in the treatment of Parkinson's disease (PD), as it reduces the extent of cell death in the substantia nigra of mice treated with the neurotoxin MPTP.

International (PCT) Patent application WO 05/123053 discloses that the (+) α-pinene derivatives exemplified by the CB₂ specific agonist HU-308 are useful in the treatment and prevention of the onset of genetic neurodegenerative disorders, in particular Huntington's disease (HD), as it reduces the extent of cell death in the basal ganglia of rats treated with intrastriatal injection of the neurotoxin malonate. Notably, the protective effect exerted by HU-308 was produced only in an environment of neuronal damage, since the compound did not alter the parameters monitored (GABA level and dopamine transmission) in the non-lesioned side. Thus, WO 05/123053 does not teach or disclose that HU-308 is effective in promoting nerve growth.

Currently, no drug exists for promoting neurogenesis and regeneration of neural tissues. Thus, it would be advantageous to provide a solution to the long-felt unmet medical need for therapeutic means of neuroregeneration.

### SUMMARY OF THE INVENTION

The present invention provides a method for promoting, inducing and enhancing neurogenesis, by administering to an individual in need thereof a pharmaceutical composition comprising a therapeutically effective amount of a CB₂ selective agonist as an active ingredient. According to the present invention it is shown for the first time that functional CB₂ receptors are expressed in neural progenitors from embryonic to adult stages and that their selective activation stimulates cell proliferation.

According to certain embodiments the CB₂ selective agonist used in the method of the invention is a cannabinoid, plant or animal derived, or a cannabimimetic compound, or analogue thereof, typically selected from the group consisting of α-pinene derivatives, aminoalkylindoles, anandamides, 3-aroylindoles, aryl and heteroaryl sulfonates, arylsulphonamides, benzamides, biphenyl-like cannabinoids, cannabinoids optionally further substituted by fused or bridged mono- or polycyclic rings, pyrazole-4-carboxamides, eicosanoids, dihydroisoindolones, dihydrooxazoles, quinazolinediones, quinolinecarboxylic acid amides, resorcinol derivatives, tetrazines, triazines, pyridazines and pyrimidine derivatives, and analogues and derivatives thereof.

According to additional embodiments the CB₂ selective agonist used in the method of the invention is a (+) or (−)-α-pinene derivative.

According to exemplary embodiments, the present invention provides a method of promoting, inducing and enhancing neurogenesis, including the step of administering to an individual in need thereof a therapeutically effective amount of a pharmaceutical composition comprising as an active ingredient a compound of formula (I):

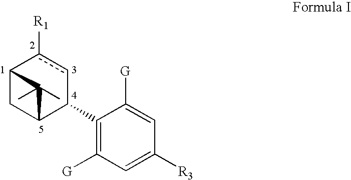

or a pharmaceutically acceptable salt, ester or solvate of said compound having a specific stereochemistry wherein C-5 is S, the protons at C-1 and C-5 are cis in relation to one another and the protons at C-4 and C-5 are trans, wherein:
the dashed line C-2-C-3 designates an optional double bond,
R₁ is selected from the group consisting of (a) —R′N(R″)₂ wherein R′ is C₁-C₅ straight or branched chain alkyl and each R″, which may be the same or different, is hydrogen or C₁-C₅ straight or branched chain alkyl optionally containing a terminal —OR′″ or —OC(O)R′″ moiety wherein R′″ is hydrogen or C₁-C₅ straight or branched chain alkyl, (b) -Q wherein Q is a heterocyclic moiety having a labile hydrogen atom so that said moiety acts as a carboxylic acid analogue, (c) —R′X wherein R′ is C₁-C₅ straight or branched chain alkyl and X is halogen, (d) —R′C(O)N(R″)₂ wherein R′ is a direct bond or C₁-C₅ straight or branched chain alkyl and each R″, which may be the same or different, is hydrogen or C₁-C₅ straight or branched chain alkyl optionally containing a terminal —OR′″ or —OC(O)R′″ moiety wherein R′″ is hydrogen or C₁-C₅ straight or branched chain alkyl, (e) —R′C(O)OR″ wherein R′ is a direct bond or C₁-C₅ straight or branched chain alkyl and R″ is hydrogen or C₁-C₅ straight or branched chain alkyl optionally containing a terminal —OR′″ or —OC(O)R′″ moiety wherein R′″ is hydrogen or C₁-C₅ straight or branched chain alkyl, (f) —R′ wherein R′ is C₁-C₅ straight or branched chain alkyl, and (g) —R′OR′″ wherein R′ is C₁-C₅ straight or branched chain alkyl and R′″ is hydrogen or C₁-C₅ straight or branched chain alkyl;
G is at each occurrence independently selected from the group consisting of hydrogen, halogen and —OR₂ wherein R₂ is hydrogen or C₁-C₅ straight or branched chain alkyl optionally containing a terminal —OR′″, —OC(O)R′″, C(O)OR′″, or —C(O)R′″ moiety wherein R′″ is hydrogen or C₁-C₅ straight or branched chain alkyl; and
R₃ is selected from the group consisting of (a) C₁-C₁₂ straight or branched chain alkyl, (b) —OR″″, in which R″″ is a straight chain or branched C₂-C₈ alkyl which may be substituted at the terminal carbon atom by a phenyl group, and (c) —(CH₂)ₙOR′″ wherein n is an integer of 1 to 7 and R′″ is hydrogen or C₁-C₅ straight or branched chain alkyl.

According to a further exemplary embodiment, the present invention provides a method of promoting, inducing and enhancing neurogenesis, including the step of administering to an individual in need thereof a prophylactically and/or therapeutically effective amount of a pharmaceutical composition comprising as an active ingredient a compound of formula (I) wherein there is a double bond between C-2 and C-3, R₁ is CH₂OH, G is OCH₃ and R₃ is 1,1-dimethylheptyl.

The ability of pharmaceutical compositions of the invention to promote, induce and enhance neurogenesis will be useful for alleviating or treating neurological injuries or damages to the CNS or the PNS associated with physical injury, ischemia, neurodegenerative disorders, certain medical procedures or medications, tumors, infections, metabolic or nutritional disorders, cognition or mood disorders, and various medical conditions associated with neural damage or destruction.

The pharmaceutical compositions used in the present invention can include in addition to the aforesaid compounds, pharmaceutically inert ingredients such as thickeners, carriers, buffers, diluents, surface active agents, preservatives and the like, all as well known in the art, necessary to produce physiologically acceptable and stable formulations.

The choice of the pharmaceutical additives, carriers, diluents, excipients and the like, will be determined in part by the particular active ingredient, as well as by the particular route of administration of the composition. The routes of administration include but are not limited to oral, aerosol, parenteral, topical, ocular, transdermal, subcutaneous, intravenous, intramuscular, intraperitoneal, intrathecal, rectal and vaginal systemic administration. In addition, the pharmaceutical compositions of the invention can be directly delivered into the CNS by intracerebroventricular, intraparenchymal, intraspinal, intracistemal or intracranial administration.

The pharmaceutical compositions can be in a liquid, aerosol or solid dosage form, and can be formulated into any suitable formulation including, but not limited to, solutions, suspensions, micelles, emulsions, microemulsions, aerosols, powders, granules, sachets, soft gels, capsules, tablets, pills, caplets, suppositories, creams, gels, pastes, foams and the like, as will be required by the particular route of administration.

Prior to their use as medicaments for treating an individual in need thereof, the pharmaceutical compositions may be formulated in unit dosage forms. The active dose for humans is generally in the range of from 0.05 mg to about 50 mg per kg body weight, in a regimen of 1-4 times a day. However, it is evident to the man skilled in the art that dosages would be determined by the attending physician, according to the disease to be treated, the method of administration, the patient's age, weight, contraindications and the like.

In another embodiment, the present invention provides use of the aforesaid compounds to promote, induce and enhance neurogenesis in vitro. The neural stem cells may be harvested from healthy tissues and cultured with compounds of the invention until a desired level of neurogenesis is achieved. The appropriate differentiation lineage and stage of maturity will depend upon the disorder to be treated. The neural cells so obtained can be used in transplant therapies of neurological disorders.

These and additional benefits and features of the invention will be better understood with reference to the following detailed description taken in conjunction with the figures and non-limiting examples.

### BRIEF DESCRIPTION OF THE FIGURES

To assist in the understanding of the invention, and in particular of the data that are given in the examples, the following drawing figures are presented herein:

FIG. 1 shows that neural progenitors express CB₂ receptors in vitro. In each case GAPDH served as internal house-keeping control in the RT-PCR experiments and α-tubulin served as internal control in the Western blots. FIG. 1A compares the level of gene expression of the CB₂ receptor and nestin in embryonic (E), postnatal (P) and adult neural progenitors as determined by RT-PCR. FIG. 1B shows the level of protein expression of the CB₂ receptor in the previously mentioned cells and tissues, as determined by Western blot. FIG. 1C shows the results of a typical immunostaining experiment of adherent embryonic and adult neural progenitor cultures, and postnatal radial glial progenitors. Scale bars 20 μm. FIG. 1D shows the analysis of CB₂ receptor expression in undifferentiated neural progenitors (NP) and their differentiated neural cell progeny (Diff NC) evaluated by the presence of nestin, β-tubulin III and GFAP transcripts.

FIG. 2 shows that neural progenitors express CB₂ receptors in vivo as assessed by confocal microscopy in adult hippocampal sections. Scale bars: 40 and 10 μm.

FIG. 3 shows that CB₂ receptors control neurosphere generation and neural progenitor cell proliferation in vitro. FIG. 3A compares the self-renewal ability of E17.5 neural progenitors derived from wild-type and CB₂^{-/-} mice. The number of neurospheres (NSP) was quantified after 5 consecutive neurosphere passages. Inset: Primary neurosphere generation in the two mouse strains (CB₂^{-/-}-white bar). FIG. 3B depicts the amount of primary neurospheres generated after 7 days of exposure of neural progenitors to the various indicated treatments. CB₂^{-/-} progenitors were also employed (where indicated). FIG. 3C shows the self-renewal ability of wild-type neural progenitors incubated with the same treatments for 5 consecutive passages, as measured by the amount of neurospheres at each passage. Self-renewal of CB₂ deficient progenitors in the presence of vehicle is also shown (as indicated). FIG. 3D shows the percentage of BrdU-positive cells from dissociated neurospheres incubated with same treatments for 16 hours. FIG. 3E shows the percentage of BrdU-positive cells (left panel) and neurosphere generation (right panel) of progenitors exposed to the indicated treatments. FIG. 3F shows ERK and Alt phosphorylation after progenitor challenge with various indicated treatments. Asterisks indicate the treatment groups that are significantly different from control wild-type cells: * P<0.05,**P<0.01.

FIG. 4 shows that CB₂ receptors control neural progenitor cell proliferation in vivo. FIG. 4A shows the number of BrdU-positive cells per section in the dentate gyrus of wild-type (WT) and CB₂^{-/-} mouse E17.5 embryos. FIG. 4B shows the number of BrdU-positive cells per section in the dentate gyrus of wild-type (WT) and CB₂^{-/-} adult mice injected with the indicated agents. FIG. 4C shows the number of BrdU-positive cells per section in the dentate gyrus of wild-type (WT) and CB₂^{-/-} adult mice (as indicated) injected with saline (Veh) or kainic acid (KA). Lower panels show representative immunostainings of BrdU-positive cells (light) co-stained with TOTO-3 (dark). Scale bars: 90 μm (A) and 45 μm (B and C). Asterisks indicate the treatment groups that are significantly different from control wild-type mice: * P<0.05, ** P<0.01. A ladder indicates a treatment group that is significantly different from knock-out mice treated with kainate: ^{#} P<0.05.

### DETAILED DESCRIPTION OF THE INVENTION

Traditional treatments of neurological diseases and injuries have focused on the prevention of neuronal death and have targeted secondary damages. In contrast, the present invention is directed to novel therapeutic treatments based on inducing neurogenesis, i.e. promoting proliferation, migration, survival and/or differentiation of neural stem cells and progenitor cells into neural cells.

The present invention provides pharmaceutical compositions and methods for promoting, inducing and enhancing neurogenesis, useful for alleviating, or treating neurological injuries or damages to the CNS or the PNS associated with physical injury, ischemia, neurodegenerative disorders, certain medical procedures or medications, tumors; infections, metabolic or nutritional disorders, cognition or mood disorders, and various medical conditions associated with neural damage or destruction. According to the present invention it is shown for the first time that functional CB₂ receptors are expressed in neural progenitors from embryonic to adult stages and that their selective activation stimulates cell proliferation.

The present invention provides methods that can be used in vivo to induce the quiescent neural stem cells of an individual in need thereof to enter neurogenesis, i.e. to grow, proliferate, migrate, survive and/or differentiate, to replace neural cells that have been damaged or destroyed and achieve in situ nerve regeneration. In another embodiment, the methods of the invention can be used in vitro to induce neural stem cells or progenitor cells harvested from the appropriate tissue to undergo neurogenesis. The cells so induced and cultured may be used for therapeutic treatment for example for transplantation into the neural tissue of an individual in need thereof in order to prevent, alleviate or treat aforesaid medical conditions.

In particular, the present invention provides pharmaceutical compositions comprising as an active ingredient CB₂ selective cannabinoid agonists and methods using the same for promoting neurogenesis, and alleviating, or treating aforesaid medical conditions.

Typically, the CB₂ selective agonist is a natural, plant derived or endogenous, or a synthetic cannabinoid selected from the group consisting of α-pinene derivatives, aminoalkylindoles, anandamides, 3-aroylindoles, aryl and heteroaryl sulfonates, arylsulphonamides, benzamides, biphenyl-like cannabinoids, cannabinoids optionally further substituted by fused or bridged mono- or polycyclic rings, pyrazole-4-carboxamides, eicosanoids, dihydroisoindolones, dihydrooxazoles, quinazolinediones, quinolinecarboxylic acid amides, resorcinol derivatives, tetrazines, triazines, pyridazines and pyrimidine derivatives, and isomers, analogues and derivatives thereof, as well as pharmaceutically acceptable salts, esters, solvates, prodrugs and polymorphs thereof. More preferably, the CB₂ selective cannabinoid agonist is a α-pinene derivative, or a mixture of a (+) and (−)-α-pinene derivative, most preferably a (+)-α-pinene derivative.

Some of the compounds according to the invention can exist in stereoisomeric forms which are either enantiomers or diastereomers of each other. The invention relates to the enantiomers or diastereomers of the compounds or mixtures thereof. These mixtures of enantiomers and diastereomers can be separated into stereoisomerically uniform components in a known manner or synthesized a priori as separate enantiomers.

### DEFINITIONS

To facilitate an understanding of the present invention, a number of terms and phrases are defined below.

As used herein, the term “central nervous system” (CNS) refers to all structures within the dura mater. Such structures include, but are not limited to, the brain and spinal cord.

As used herein, the term “peripheral nervous system” (PNS) refers to all other neural elements outside the brain and the spinal cord, and it includes nerves, ganglia, spinal and cranial nerves.

The neuron is the basic building block of the nervous system, both CNS and PNS, where it receives, processes and transmits electrical information from one part of the body to another. A neuron consists of a cell body and two or more extensions, called dendrites and axons. Dendrites receive inputs and conduct signals toward the cell body, whereas axons conduct signal away from the body to other neurons or target cells to which they connect.

As used herein, the term “CB” refers to cannabinoid receptors. CB₁ receptors are predominantly found in the CNS, whereas CB₂ receptors are predominantly found in the periphery on immune cells. Aside from these two receptors, evidence exists supporting the presence of yet uncloned cannabinoid receptors.

In the present invention, binding affinity is represented by the IC₅₀ value, namely the concentration of a test compound that will displace 50% of a radiolabeled agonist from the CB receptors. Preferred compounds display IC₅₀ values for CB₂ binding of 50 nM or lower, preferably of 30 nM or lower, more preferably of 10 nM or lower and most preferably of 1 nM or lower. “CB₂ specific or selective” denotes compounds with a ratio of CB₂/CB₁ binding affinity that is at least 10, preferably 20, more preferably 50 and most preferably 100 or greater. Preferably these ratios will be obtained for human CB₁ and CB₂ receptors. The selectivity toward CB₂, denoted CB₂/CB₁ affinity, is calculated as the IC₅₀ value obtained by the test compound for the displacement of the CB₁ specific radioligand divided by the IC₅₀ value obtained for the displacement of the CB₂ specific radioligand, i.e. the IC₅₀ CB₁/IC₅₀ CB₂. Some of the preferred compounds of the present invention do not necessarily share both properties, in other words some have an IC₅₀ ratio of 100 or greater for CB₂/CB₁ affinity and an IC₅₀ for CB₂ of only about 10 nM.

An agonist is a substance that mimics a specific ligand, for example a hormone, a neurotransmitter, or in the present case a cannabinoid, able to attach to that ligand's receptor and thereby produce the same action that the ligand produces. Though most agonists act through direct binding to the relevant receptor and subsequent activation, some agonists act by promoting the binding of the ligand or increasing its time of residence on the receptor, increasing the probability and effect of each coupling. Whatever the mechanism of action, all encompassed in the present invention, the net effect of an agonist is to promote the action of the original chemical substance serving as ligand. Compounds that have the opposite effect, and instead of promoting the action of a ligand, block it are receptor antagonists.

### Neurogenesis

During mammalian development the generation of the central nervous system relies on a finely regulated balance of neural progenitor proliferation, differentiation and survival that is controlled by a number of extracellular signaling cues [Alvarez-Buylla, A. and Lim, D., Neuron 41, 683-6, 2004; Lie, D. C. et al., Annu. Rev. Pharmacol. Toxicol. 44, 399-421, 2004]. Throughout history, scientists have commonly believed that once the brain is damaged, either through accident, disease or aging, there is no way to repair it. However, in the past few years, neuroscientists have discovered that the brain does change throughout life, and can possibly repair itself. Some of this repair occurs through neurogenesis, or the birth of new neuronal cells from neural stem cells and progenitors. Neurogenesis has been recently demonstrated to occur not only during development, but throughout adult life. The existence of hippocampal neurogenesis in the adult brain has received strong support by the identification of a neural progenitor cell population located in the subgranular zone [Gotz, M. and Huttner, W.B., Nat. Rev. Mol. Cell. Biol. 6, 777-88, 2005]. These neural progenitors give raise to newly generated cells that can integrate properly in hippocampal circuits and thus may contribute to synaptic plasticity [Santarelli, L. et al., Science 301, 805-9, 2003], enabling organisms to adapt to environmental changes, cognitive functions, influencing learning and memory, and neuroregeneration upon brain damage [Nakatomi, H. et al., Cell 110, 429-41, 2002]. Neurogenesis and its promotion would be useful for the treatment of numerous diseases or disorders wherein nerves are damaged.

In order for new brain cells to develop, multipotent neural stem cells (NSCs) divide in the brain and develop into any of the three basic cell types of the CNS: neurons, oligodendrocytes and astrocytes. Following a given signal, which could be triggered by adverse events, neural stem and progenitor cells proliferate, migrate from proliferative regions to sites of neurogenesis or injury and differentiate into mature cells upon connection with other neurons. The stem cells, mostly quiescent, are undifferentiated cells that exhibit the ability to proliferate, self-renew, and to differentiate into multiple yet distinct lineages. In contrast, progenitor cells are mitotic cells with a faster dividing cell cycle that retain limited ability to proliferate and to give rise to terminally differentiated cells. Progenitors are more committed than stem-like cells and they are not capable of indefinite self-renewal. Progenitor cells are also referred to as precursors and their multipotentiality is still being debated.

Not all multipotent cells entering this process survive till its completion. It takes over a month for the new neuron to be able to send and receive messages, showing that neurogenesis is a controlled process. Neurogenesis is regulated by growth factors that can lead to the development of new cells. Once the cells become either glial cells or neurons, other growth factors including brain-derived neurotrophic factor participate in their maturation and survival. It would be advantageous to understand the mechanisms underlying neurogenesis in order to identify the molecules which could be used to promote this process and enhance neural regeneration. Clearly therapies that could increase neural regeneration that might ultimately lead to partial or full functional recovery, and may also help to palliate injury-associated symptoms, would be highly beneficial to patients and would significantly reduce health care costs.

As used herein, the term “neurogenesis” refers to the process by which neurons are created. Neurogenesis encompasses proliferation of neural stem and progenitor cells, differentiation of these cells into new neural cell types, as well as migration and survival of the new cells. The term is intended to cover neurogenesis as it occurs during normal development, predominantly during pre-natal and peri-natal development, as well as neural cells regeneration that occurs following disease, damage or therapeutic intervention. Adult neurogenesis is also termed “nerve” or “neural” regeneration.

As used herein, the term “neurosphere(s)” refers to neural stem and progenitor cells that were expanded in vitro, and it includes both the free-floating aggregates and the dissociated individual cells. The neurospheres comprise heterogeneous cell populations at various developmental stages.

It is understood that the neuroregenerative properties of compounds of the invention refer to events wherein the neurons are actively stimulated or promoted to regrow or regenerate in a maimer that will achieve improvement or repair of neuronal circuits within damaged neural tissues, but which are distinct from passive neuroprotective treatments which prevent neuronal cell death. Traditional neuroprotection, if administered within a rather limited temporal window following insult, can only prevent further degeneration and does not repair damaged neural tissues. In contrast, neurogenesis can be induced even at time points remote from initial injury, it can be stimulated in vivo or in vitro for later reimplantation, and it could ultimately repair damaged tissues.

### In Vitro Neurogenesis

The invention provides a method of promoting, inducing and enhancing neurogenesis in vivo wherein neural cells damaged by injury, therapy or disease, are endogenously replaced. In addition, the present invention provides a method to induce neurogenesis in vitro. The neural cells obtained by such methods, which can be derived from heterologous or autologous host, can be used in transplantation therapy for individuals suffering from neurological disorders. Multipotent stem cells can be obtained from embryonic, postnatal, juvenile or adult neural tissues. Embryonic cells may be derived from fetal tissue following elective abortion, other cells can be obtained from donors or by biopsy.

Procedures for culturing neural cells are well known. Proliferation and differentiation are monitored by methods known in the art, some of which will be exemplified herein-below. For instance cellular differentiation may be monitored by using antibodies to antigens specific for neurons, astrocytes or oligodendrocytes, and assessed by immunocytochemistry techniques. Additional analysis may be performed by in situ hybridization histochemistry, Western, Southern and Northern blot procedures, using standard molecular biology techniques.

Following in vitro expansion and neurogenesis using a method of the invention, the cells can be administered to an individual with abnormal neurological or neurodegenerative symptoms.

### Therapeutic Uses of Neurogenesis

Pharmacological agents able to promote, induce and enhance neurogenesis will be useful in methods of preventing, alleviating or treating diseases or disorders wherein nerves of the CNS or the PNS are damaged due to physical injury, ischemia, neurological disorders, certain medical procedures or medications, tumors, infections, metabolic or nutritional disorders, cognition or mood disorders, and various medical conditions associated with neural damage or destruction.

A list of diseases and disorders associated with the nervous system, which could profit from neurogenesis, either induced in vivo or in vitro as a preliminary step to transplantation, can be found at the web site of the National Institute of Neurological Disorders and Stroke (NINDS), which is part of the National Institutes of Health (NIH) at http://www.ninds.nih.gov/disorders/disorder_index.htm.

Compositions of the invention will be useful in promoting, inducing and enhancing neurogenesis in the physically injured nervous system of a subject. Such injuries include, but are not limited to, head trauma, mild to severe traumatic brain injury (TBI), spinal cord injury, diffuse axonal injury and other forms of craniocerebral trauma such as cranial nerve injuries, cerebral contusion, intracerebral haemorrhage and acute brain swelling.

Compositions of the invention will be useful when the nerves are damaged as a result from certain medical procedures, including, but not limited to, surgery which compromise oxygen delivery to the brain such as coronary artery bypass graft (CABG), electroconvulsive therapy, radio- or chemotherapy. Certain medications or other chemical agents are known to cause some level of neurodegeneration and compounds of the invention can be used to promote neurogenesis in cases where a subject was exposed to alcohol, psychoactive, sedative or hypnotic drugs, bacterial or industrial toxins, lead, plant poisons, venomous bites and stings, anti-neoplastic and immunosuppressive agents, and the like as known to medical practitioners.

Compositions of the invention will be useful when the nerve damage results from ischemia including, but not limited to, spinal cord infarction or ischemia, ischemic infarction, stroke, cardiac insufficiency or arrest, atherosclerotic thrombosis, ruptured aneurysm, embolism and haemorrhage, such as hypotensive or hypertensive haemorrhage.

Compositions of the invention will be useful when the nerve damage results from tumors, including, but not limited to, CNS metastasis, intraaxial tumors such as primary CNS lymphomas, germ cell tumors, infiltrating and localized gliomas, fibrillary astrocytomas, oligodendrogliomas, ependymomas, pleomorphic xanthoastrocytomas, pilocytic astrocytomas; extraaxial brain tumors that arise in the spinal and cranial nerves such as meningiomas, schwannomas, neurofibromas, pituitary tumors as well as mesenchymal tumors of the skull, spine and dura matter.

Compositions of the invention will be useful when the nerve damage results from infections of bacterial, viral, fungal, parasitic or other origin, including, but not limited to, pyrogenic infections, meningitis, tuberculosis, syphilis, encephalomyelitis and leptomeningitis.

Compositions of the invention will be useful when the nerve damage results from metabolic or nutritional disorders, including, but not limited to, glycogen storage diseases, acid lipase diseases, Wemicke's or Marchiafava-Bignami's disease, Lesch-Nyhan syndrome, Farber's disease, gangliosidoses, vitamin B12 and folic acid deficiency.

Compositions of the invention will be useful when the nerve damage results from neurodegenerative disorders, including, but not limited to, Alzheimer's disease (AD), Lewy Body dementia, Parkinson's disease (PD), Huntington's disease (HD), non-Huntingtonian type of Chorea, Pick's disease, Creutzfeldt-Jakob disease (CJD), kuru, Guillain-Barré syndrome, progressive supranuclear palsy; or neurological lesions associated with diabetic neuropathy, Bell's palsy, systemic lupus erythematosius (SLE), demyelinating disorders, amyotrophic lateral sclerosis (ALS), multiple sclerosis (MS), motor neuron disease, retinal degeneration, muscular dystrophy, Dejerine-Sottas syndrome and peripheral neuropathies.

Compositions of the invention will be useful to prevent, alleviate or treat other medical conditions where neurons are damaged or destroyed, including, but not limited to, asphyxia, prematurity in infants, perinatal distress, gaseous intoxication for instance from carbon monoxide or ammonia, coma, hypoglycaemia, dementia, epilepsy and hypertensive crises.

In their recent review on adult neurogenesis, Abrous et al. have discussed some of the pathologies associated with abnormal neurogenesis [Abrous, D. N. et al., Physiol. Rev. 85, 523-69, 2005]. Interestingly, they have reported that the activity of certain therapeutic agents, such as antidepressants, is now believed to be at least partly mediated by their ability to stimulate neurogenesis. Therefore, in view of their safety, compounds of the invention might advantageously replace existing medications having known side effects and provide alternative strategies for the treatment of mood disorders. It has been reported that chronic stress induces structural changes in neuronal networks, in particular in the hippocampus, the prefrontal cortex and the amygdale and inhibits adult neurogenesis in the dentate gyrus.

Because neurogenesis is involved in learning and memory, compounds of the invention can also be used in normal individuals to enhance learning and/or memory, or to treat individuals with cognitive disorders. The cognitive deficits could be associated with diseases or age-related.

### Cannabinoids and Neurogenesis

To date, the effects of endocannabinoids on the modulation of synaptic plasticity and neuronal excitability, as well as of neural cell survival [Mechoulam, R. et al., Sci. STKE 129, RE5, 2002; Guzman, M., Nat. Rev. Cancer 3, 745-7, 2003], have been attributed solely to the engagement of “central” CB₁ receptors. The expression pattern of the CB₁ receptor is regulated during brain development [Fernandez-Ruiz, J. et al., Trends Neurosci. 23, 14-20, 2001], and the receptor remains expressed at high levels in differentiated neurons and at lower levels in glial cells of various adult brain areas such as the hippocampus, basal ganglia and cortex. In contrast, the presence of the “peripheral” CB₂ receptor in differentiated neurons and glial cells is more restricted. Thus, only recently the expression of CB₂ receptors in normal brain could be demonstrated in the cerebellum as well as in a subpopulation of neurons of the vagus nerve in the brainstem, where it participates in the regulation of emesis. In addition, CB₂ receptor expression in the brain is also found in microglia and endothelial cells.

The first study that attempted to correlate cannabinoids to neurogenesis was performed by Jiang et al. The observed activity, restricted to proliferation and excluding differentiation, was attributed to the agonistic properties of the test compound toward CB₁.

In the present invention it is shown for the first time that functional CB₂ receptors are expressed in neural progenitors from embryonic to adult stages and that their selective activation stimulates cell proliferation. Interestingly, other studies had previously suggested an inverse relation between CB₂ receptor expression and stage of cell differentiation. For example, CB₂ receptor expression decreases during B-cell differentiation [Carayon, P. et al., Blood 92, 3605-15, 1998] and increases with dedifferentiation (i.e. with increased malignancy) of glial tumors [Sanchez, C. et al., Cancer Res. 61, 5784-5789, 2001]. Likewise, CB₂ receptor activation and overexpression [Alberich Jorda, M. et al., Blood 104, 526-34, 2004] block neutrophil cell differentiation. Thus, without wishing to be bound by any theory or particular mechanism of action cannabinoids may control neural progenitor cell function via CB₂ receptors acting as a “cell dedifferentiation signal” by favouring a non-differentiated, proliferative state.

Thus CB₂-selective ligands provide pharmacological agents now disclosed to be able to modulate neural progenitor cell fate. In addition, CB₂-selective agonists are attractive therapeutic agents as they do not cause CB₁-mediated psychoactive effects.

CB₂ receptor expression in brain has been partially examined in differentiated cells, while its presence and function in neural progenitor cells remained unknown. It is now shown, as detailed below in Example 1, that the CB₂ receptor is expressed, both in vitro and in vivo, in neural progenitors from late embryonic stages to adult brain. In addition, it is demonstrated, as detailed below in Example 2, that selective pharmacological activation of the CB₂ receptor in vitro promotes neural progenitor cell proliferation and neurosphere generation, an action that is impaired in CB₂-deficient cells. Accordingly, in vivo experiments, detailed below in Example 3, evidence that hippocampal progenitor proliferation is increased by administration of the CB₂-selective agonist HU-308. Moreover, impaired progenitor proliferation was observed in CB₂-deficient mice both in normal conditions and upon kainate-induced excitotoxicity. These findings, which demonstrate in vitro and in vivo neurogenesis, provide a novel physiological role for the CB₂ cannabinoid receptor and open a novel therapeutic avenue for manipulating neural progenitor cell fate.

The present invention provides use of CB₂ agonists for the promotion of neural regeneration, as exemplified herein below with known CB₂ specific agonist HU-308, the full chemical name of which is (+) {4-[4-(1,1-dimethylheptyl)-2,6-dimethoxyphenyl]-6,6-dimethyl-bicyclo[3.1.1]hept-2-en-2-yl}-methanol, also disclosed in WO 01/32169 as (+) 4-[2,6-dimethoxy-4-(1,1-dimethyl-heptyl)-phenyl]-6,6-dimethyl-bicyclo[3.1.1]hept-2-ene-2-carbinol. As disclosed in WO 03/064359, HU-308 binds human CB₂ receptors with an IC₅₀ of 13.3 nM and human CB₁ receptors with an IC₅₀ of 3600 nM, yielding a selectivity of about 270 fold for CB₂ binding affinity over CB₁.

### Suitable CB₂ Selective Agonist Compounds

Suitable cannabinoid analogues are disclosed in U.S. Pat. No. 6,017,919 to Inaba et al. and in U.S. Pat. No. 6,166,066 to Makriyannis et al., the contents of which are hereby incorporated herein by reference in their entirety These compounds include acrylamide derivatives, benzamides, dihydroisoindolones, isoquinolinones, and quinazolinediones, as well as pentyloxyquinolines, dihydrooxazoles and non-classical cannabinoids in which the alkyl chain typically found in cannabinoids has been replaced with a monocyclic or bicyclic ring that is fused to the tricyclic core of classical cannabinoids.

United States Patent Applications Nos. 2004/0087590, 2004/0077851, 2004/0077649, 2003/0120094 and 2001/0009965 to Makriyannis et al., 2004/0034090 to Barth et al., 2003/0232802 to Heil et al., 2003/0073727 to Mittendorf et al., and 2002/0077322 to Ayoub, the contents of which are hereby incorporated herein by reference in their entirety, disclose a number of cannabinoid analogues suitable for use in the methods according to the present invention. These compounds include biphenyl and biphenyl-like cannabinoids, aminoalkylindoles, heterocyclic compounds including tetrazines, triazines, pyridazines and pyrimidine derivatives, 3-aroylindoles, aryl and heteroaryl sulfonates, arylsulphonamides and cannabinoids with a monocyclic, fused bicyclic, a bridged bicyclic or a bridged tricyclic side chain at the C-3 position of the phenyl ring of classical cannabinoids.

PCT Patent Application No. WO 03/091189 to Martin et al., incorporated herein by reference in its entirety, discloses a number of resorcinol derivatives suitable for use in the methods according to the present invention.

U.S. Pat. No. 4,208,351 to Archer et al. and PCT Patent Applications Nos. WO 01/28497 and WO 03/005960 to Makriyannis et al., WO 01/32169 to Fride et al., and WO 03/064359 and WO 03/063758 to Garzon et al., the contents of which are incorporated herein by reference in their entirety, disclose a number of classical and non-classical cannabinoid analogues suitable for use in the methods according to the present invention. These compounds include classical Δ⁹-THC type of compounds and bicyclic (−) and (+)-α-pinene derivatives.

In general, it has been possible to functionally differentiate between the R and S enantiomers of cannabinoid and cannabinoid-related compounds. The compounds HU-210 and HU-211 exemplify this. HU-210 is the (−)(3R,4R) enantiomer of the synthetic cannabinoid, 7-hydroxy-Δ⁶-tetrahydrocannabinol-1,1-dimethyl-heptyl. HU-211 is the (+)(3S,4S) enantiomer of this compound. In contrast to HU-210, HU-211 exhibits low affinity to the cannabinoid receptors and is thus non-psychotropic. In addition, it functions as a noncompetitive NMDA-receptor antagonist and as a neuroprotective agent, two properties absent in HU-210 (See, U.S. Pat. No. 5,284,867).

### α-Pinene Compounds

The numbering of positions in the ring structure shown below is used to describe the α-pinene compounds used in the methods of the present invention. Positions 1, 4 and 5 are chiral centers. The stereochemistry of the preferred (+)-α-pinene derivatives is such that C-5 is the (S) configuration, the protons at C-1 and C-5 are cis in relation to one another and the protons at C-4 and C-5 are trans in relation to one another as shown in formula (II):

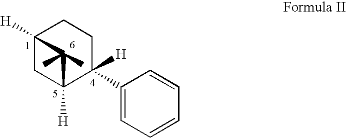

The stereochemistry of the (−)-α-pinene derivatives disclosed in the present invention is such that C-5 is in the (R) configuration, the protons at C-1 and C-5 are cis in relation to one another and the protons at C-4 and C-5 are trans in relation to one another.

### CHEMICAL DEFINITIONS

Throughout this specification, certain compounds of the present invention can be referred to by capital letters followed by numbers, e.g. HU-308, rather than by their full chemical names. The alkyl substituents can be saturated or unsaturated (e.g. alkenyl, allynyl), linear, branched or cyclic, the latter only when the number of carbon atoms in the alkyl chain is greater than or equal to three. When unsaturated, the hydrocarbon radicals can have one double bond or more and form alkenyls, or one triple bond or more and form alkynyls. Regardless of the degree of unsaturation, all of the alkyl substituents can be linear or branched.

OR represents hydroxyl or ethers, OC(O)R and C(O)OR represent esters, C(O)R represents ketones, C(O)NR₂ represents amides, NR₂ represents amines, wherein R is a hydrogen or an alkyl chain as defined above.

“Halogen” or “halo” means fluorine (—F), chlorine (—Cl), bromine (—Br) or iodine (—I) and if the compound contains more than one halogen (e.g., two or more variable groups can be a halogen), each halogen is independently selected from the aforementioned halogen atoms.

The term “substituted” or “optionally substituted” means that one or more hydrogens on the designated atom is replaced or optionally replaced with a selection from the indicated group, provided that the designated atom's normal valency under the existing circumstances is not exceeded. Combination of substituents and/or variables are permissible only if such combinations result in stable compounds. By “stable compound” or “stable structure” is meant a compound that is sufficiently robust to survive isolation to a useful degree of purity from a reaction mixture, and formulation into an efficacious therapeutic agent.

### Pharmaceutically Acceptable Compounds

The present invention also includes within its scope solvates of compounds of formula (I) and salts thereof. “Solvate” means a physical association of a compound of the invention with one or more solvent molecules. This physical association involves varying degrees of ionic bonding, including hydrogen bonding. In certain instances the solvate will be capable of isolation. “Solvate” encompasses both solution-phase and isolatable solvates. Non-limiting examples of suitable solvates include alcohol solvates such as ethanolates, methanolates and the like. “Hydrate” is a solvate wherein the solvent molecule is water.

The term “polymorph” refers to a particular crystalline state of a substance, which can be characterized by particular physical properties such as X-ray diffraction, IR spectra, melting point, and the like.

In the present specification the term “prodrug” represents compounds which are rapidly transformed in vivo to parent compound of formula (I), for example by hydrolysis in the blood. Prodrugs are often useful because in some instances they can be easier to administer than the parent drug. They can, for instance, be bioavailable by oral administration whereas the parent drug is not. The prodrug can also have improved solubility compared to the parent drug in pharmaceutical compositions. All of these pharmaceutical forms are intended to be included within the scope of the present invention.

Certain compounds of the invention are capable of further forming pharmaceutically acceptable salts and esters. “Pharmaceutically acceptable salts and esters” means any salt and ester that is pharmaceutically acceptable, that is pharmacologically tolerated, and that has the desired pharmacological properties. Such salts, formed for instance by any carboxy group present in the molecule, include salts that can be derived from an inorganic or organic acid, or an inorganic or organic base, including amino acids, which is not toxic or otherwise unacceptable.

Pharmaceutically acceptable acid addition salts of the compounds include salts derived from inorganic acids such as hydrochloric, nitric, phosphoric, sulfuric, hydrobromic, hydriodic, phosphorous, and the like, as well as salts derived from organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyrophosphate, chloride, bromide, iodide, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate or galacturonate [Berge S. M. et al., J. of Pharmaceutical Science 66, 1-19, 1977].

The acid addition salts of said basic compounds are prepared by contacting the free base form with a sufficient amount of the desired acid to produce the salt in the conventional manner. The free base form can be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

The base addition salts of the acidic compounds are prepared by contacting the free acid form with a sufficient amount of the desired base to produce the salt in the conventional manner. The free acid form can be regenerated by contacting the salt form with an acid and isolating the free acid in a conventional manner. The free acid forms differ from their respective salt forms somewhat in certain physical properties such as solubility in polar solvents, but otherwise the salts are equivalent to their respective free acid for purposes of the present invention.

### Pharmacology

In the present specification and claims which follow the term “prophylactically effective” refers to the amount of compound which will achieve the goal of prevention of onset, reduction or eradication of the risk of occurrence of the disorder, in the present case neurodegeneration, while avoiding adverse side effects. Compounds of the invention can be used as preventive agents for example before carrying out medical procedures associated with neurodegeneration, including but not limited to elective surgery, electroconvulsive therapy, radiotherapy or chemotherapy.

The term “therapeutically effective” refers to the amount of compound that will achieve, with no or few adverse effects, alleviation, diminished progression or treatment of the disorder, once the disorder cannot be further delayed and the patients are no longer asymptomatic, hence providing either a subjective relief of a symptom(s) or an objectively identifiable improvement as noted by the clinician or other qualified observer. The compositions of the present invention are prophylactic as well as therapeutic and treating or alleviating the disease is explicitly meant to include preventing or delaying the onset of the disease.

An “effective amount”, whether prophylactic or therapeutic, is the amount of compound sufficient to achieve a statistically significant promotion of neurogenesis compared to a control. Nerve cell growth or nerve regeneration can be readily assessed in in vitro or in vivo assays. Preferably the promotion of neurogenesis will achieve an increase in nerve cell growth or regeneration of at least 10%, more preferably at least 30% and most preferably 50% or more compared to control.

The “individual” or “patient” for purposes of treatment includes any human or animal affected by any of the diseases where the treatment has beneficial therapeutic impact. Usually, the animal is a vertebrate such as a primate including chimpanzees, monkeys and macaques, a rodent including mice, rats, ferrets, rabbits and hamsters, a domestic or game animal including bovine species, equine species, pigs, sheep, caprine species, feline species, canine species, avian species, and fishes.

Hereinafter, the term “oral administration” includes, but is not limited to, administration by mouth for absorption through the gastrointestinal tract (peroral) wherein the drug is swallowed, or for trans-mucosal absorption in the oral cavity by buccal, gingival, lingual, sublingual and oro-pharyngeal administration. Compositions for oral administration include powders or granules, suspensions or solutions in water or non-aqueous media, sachets, capsules or tablets. The oral composition can optionally contain inert pharmaceutical excipients such as thickeners, diluents, flavorings, dispersing aids, emulsifiers, binders, preservatives and the like.

The term “parenteral administration” as used herein indicates any route of administration other than via oral administration and includes, but is not limited to, administration by intravenous drip or bolus injection, intraperitoneal, intrathecal, subcutaneous, or intra muscular injection, topical, transdermal, rectal, nasal administration or by inhalation.

Formulations for parenteral administration include but are not limited to sterile aqueous solutions which can also contain buffers, diluents and other suitable additives.

In addition, the compositions described herein can be directly delivered to the CNS by intracerebroventricular, intraparenchymal, intraspinal, intracisternal or intracranial administration.

The compositions described herein are suitable for administration in immediate release formulations, and/or in controlled or sustained release formulations. The sustained release systems can be tailored for administration according to any one of the proposed administration regimes. Slow or extended-release delivery systems, including any of a number of biopolymers (biological-based systems), systems employing liposomes, and polymeric delivery systems, can be utilized with the compositions described herein to provide a continuous or long-term source of therapeutic compound(s).

It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

The pharmaceutical compositions can contain in addition to the active ingredient conventional pharmaceutically acceptable carriers, diluents and excipients necessary to produce a physiologically acceptable and stable formulation. The terms carrier, diluent or excipient mean an ingredient that is compatible with the other ingredients of the compositions disclosed herein, especially substances which do not react with the compounds of the invention and are not overly deleterious to the patient or animal to which the formulation is to be administered. For compounds having poor solubility, and for some compounds of the present invention that are characteristically hydrophobic and practically insoluble in water with high lipophilicity, as expressed by their high octanol/water partition coefficient and log P values, formulation strategies to prepare acceptable dosage forms will be applied. Enabling therapeutically effective and convenient administration of the compounds of the present invention is an integral part of this invention.

The pharmaceutical compositions can be in a liquid, aerosol or solid dosage form, and can be formulated into any suitable formulation including, but not limited to, solutions, suspensions, micelles, emulsions, microemulsions, aerosols, ointments, gels, suppositories, capsules, tablets, and the like, as will be required for the appropriate route of administration.

Solid compositions for oral administration such as tablets, pills, capsules, soft gels or the like can be prepared by mixing the active ingredient with conventional, pharmaceutically acceptable ingredients such as corn starch, lactose, sucrose, mannitol, sorbitol, talc, polyvinylpyrrolidone, polyethyleneglycol, cyclodextrins, dextrans, glycerol, polyglycolized glycerides, tocopheryl polyethyleneglycol succinate, sodium lauryl sulfate, polyethoxylated castor oils, non-ionic surfactants, stearic acid, magnesium stearate, dicalcium phosphate and gums as pharmaceutically acceptable diluents. The tablets or pills can be coated or otherwise compounded with pharmaceutically acceptable materials known in the art, such as microcrystalline cellulose and cellulose derivatives such as hydroxypropylmethylcellulose (HPMC), to provide a dosage form affording prolonged action or sustained release. Coating formulations can be chosen to provide controlled or sustained release of the drug, as is known in the art.

Other solid compositions can be prepared such as suppositories or retention enemas, for rectal administration using conventional suppository bases such as cocoa butter or other glycerides. Liquid forms can be prepared for oral administration or for injection, the term including but not limited to subcutaneous, transdermal, intravenous, intraperitoneal, intrathecal, and other parenteral routes of administration. The liquid compositions include aqueous solutions, with or without organic cosolvents, aqueous or oil suspensions including but not limited to cyclodextrins as suspending agent, flavored emulsions with edible oils, triglycerides and phospholipids, as well as elixirs and similar pharmaceutical vehicles. In addition, the compositions of the present invention can be formed as aerosols, for intranasal and like administration. For administration by inhalation, the compounds of the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch. Topical pharmaceutical compositions of the present invention can be formulated as solution, lotion, gel, cream, ointment, emulsion or adhesive film with pharmaceutically acceptable excipients including but not limited to propylene glycol, phospholipids, monoglycerides, diglycerides, triglycerides, polysorbates, surfactants, hydrogels, petrolatum or other such excipients as are known in the art.

Pharmaceutical compositions of the present invention can be manufactured by processes well known in the art, e.g., by means of conventional mixing, dissolving, granulating, dry-mixing, direct compression, grinding, pulverizing, dragee-making, levigating, emulsifying, encapsulating, entrapping or lyophilizing processes.

Prior to their use as medicaments, the pharmaceutical compositions will generally be formulated in unit dosage forms. The active dose for humans can be determined by standard clinical techniques and is generally in the range of from 0.01 mg to about 50 mg per kg body weight, in a regimen of 1-4 times a day. The preferred range of dosage varies with the specific compound used and is generally in the range of from about 0.1 mg to about 20 mg per kg body weight. However, it is evident to one skilled in the art that dosages would be determined by the attending physician, according to the disease or disorder to be treated, its severity, the desired therapeutic effect, the duration of treatment, the method and frequency of administration, the patient's age, weight, gender and medical condition, concurrent treatment, if any, i.e. co-administration and combination with additional medications, contraindications, the route of administration, and the like. The administration of the compositions of the present invention to a subject in need thereof can be continuous, for example once, twice or thrice daily, or intermittent for example once weekly, twice weekly, once monthly and the like, and can be gradual or continuous, constant or at a controlled rate.

Effective doses can be extrapolated from dose-response curves derived from in vitro or animal model test systems. For example, an estimated effective mg/kg dose for humans can be obtained based on data generated from mice or rat studies, for an initial approximation the effective mg/kg dosage in mice or rats is divided by twelve or six, respectively.

### EXAMPLES

### Materials

The following materials were kindly donated: the CB₂ selective agonist HU-308 by Pharmos (Rehovot, Israel) [Hanus, L. et al., Proc. Natl. Acad. Sci. USA 96, 14228-33, 1999], JWH-133 by John W. Huffman (Clemson University, Clemson, N.C.) [Huffman, J. W. et al., Bioorg. Med. Chem. 7, 2905-14, 1999], the CB₂ selective antagonist SR144528 by Sanofi-Aventis (Montpellier, France) and anti-mouse phosphorylated-S55 vimentin monoclonal 4A4 antibody by Verónica Cerdefño (University of California San Francisco, Calif.). Anti-CB₂ receptor polyclonal antibody was purchased from Affinity Bioreagents (Colorado, USA). Mouse monoclonal anti-nestin antibody was from Chemicon, and mouse monoclonal anti-NeuN, anti-GFAP, anti-α-tubulin antibodies were from Sigma. Rat monoclonal anti-BrdU antibody was from Abcam (Cambridge, UK) and monoclonal anti-RC2 antibody was from the Developmental Studies Hybridoma Bank (Iowa City). Sheep polyclonal anti-phosphoY180-ERK1/2 was from Upstate Biotechnology (Lake Placid, N.Y.) and rabbit polyclonal anti-Akt, phosphoS473-Akt and anti-ERK1/2 were from Cell Signalling Technology (Beverly, Mass.). PD98059 and LY294,002 were from Alexis Biochemicals (San Diego, Calif.). Unless otherwise stated, purchased reagents were used according to supplier's instructions.

Unless otherwise indicated, the test compounds were prepared as follows: the compounds were first dissolved and stepwise diluted in dimethylsulfoxide (DMSO) and then diluted in the assay buffer, generally tissue culture medium, down to a final concentration of 0.1% DMSO (v/v). Control incubations included the corresponding vehicle content and no significant influence of DMSO on any of the parameters determined was observed at the final concentration used.

### Animals

Wild type mice and CB₂ receptor knock-out mice (background: C57BL6) were kindly provided by Nancy Buckley (National Institute of Health, Bethesda, Md.) [Buckley, N. E. et al., Eur. J. Pharmacol. 396, 141-9, 2000]. Animal procedures were performed according to the European Union guidelines (86/609/EU) for the use of laboratory animals. Unless otherwise stated, animals were acclimated one week before initiation of study, and maintained under controlled environment. Animals were housed, at most 10 per cage, on a 12 hours light/12 hours dark regimen, at a constant temperature of 22±4° C. and controlled humidity of 55±15% RH, with pellets of rodent diet and drinking filtered water ad libitum. The animals were sacrificed at the indicated developmental stage with an i.p. injection of 100 mg/kg sodium pentobarbitone (CTS).

### Methods

### Neurosphere and Neural Progenitor Cell Culture

Multipotent self-renewing progenitors were obtained from the dissected cortices of mice at the indicated developmental stages and grown in chemically-defined medium constituted by Dulbecco's modified Eagle's and F12 media supplemented with N2 (Invitrogen), 0.6% glucose, non-essential amino acids, 50 mM Hepes, 2 μg/ml heparin, 20 ng/ml epidermal growth factor and 20 ng/ml basic fibroblast growth factor [Aguado, T. et al., J. Neurosci. 26, 1551-61, 2006]. Clonal neurospheres were cultured at 1000 cells/ml, dissociated with accutase (Sigma-Aldrich, Missouri, USA) and experiments were carried out with early (up to 10) passage neurospheres. Neurosphere generation experiments were performed in 96-well dishes with 100 μl of medium, and the number of neurospheres was quantified at predetermined time points by phase-contrast microscopy. Embryonic neural progenitors from wild-type and CB₂-deficient mice were cultured (10,000 cells/ml) in the continuous presence of cannabinoids or controls for the indicated number of passages (one passage every 4 days).

Adult neural progenitors were obtained from hippocampi of 4-month-old adult mice and cultured as described above. Neural progenitor cell differentiation was performed as described.

### Cell Proliferation Assays

These studies involve the use of bromodeoxyuridine (BrdU), a thymidine analog incorporated into DNA during the S phase of the cell cycle, which allows visualizing cell proliferation. Neural progenitor proliferation was determined by quantifying BrdU-positive cells 16 hours after incubation with 10 μg/ml BrdU, followed by immunostaining [Aguado, T. et al., FASEB J. 19, 1704-6, 2005].

The CB₂ selective cannabinoids HU-308 and JWH-133, both at a concentration of 30 nM, either alone or in combination with 2 μM of the CB₂ antagonist SR144528 were added at the beginning of the experiment and coincubated with the cells until proliferation was assessed.

Results are expressed as percentage of BrdU-positive cells over total cells.

### Western Blot

Identical protein amounts of cleared cell extracts were subjected to SDS-PAGE, transferred to PVDF membranes, and following antibody incubations developed with enhanced chemiluminiscence detection kit [Aguado, T. et al., FASEB J. 19, 1704-6, 2005]. Loading controls were performed with an anti-α-tubulin antibody.

### RT-PCR

RNA was obtained with the RNeasy Protect kit (Quiagen) using the RNase-free DNase kit. cDNA was subsequently obtained using the Superscript First-Strand cDNA synthesis kit (Roche) and amplification of cDNA was performed with the following primers:

| | |
|---|---|
| mouse CB₂ sense | |
| GGATGCCGGGAGACAGAAGTGA | (Seq. ID No. 1) |
| mouse CB₂ antisense | |
| CCCATGAGCGGCAGGTAAGAAAT | (Seq. ID No. 2) |
| human CB₂ sense | |
| CAACCCAAAGCCTTCTAGACAAG | (Seq. ID No. 3) |
| human CB₂ antisense | |
| GTGGATAGCGCAGGCAGAGGT | (Seq. ID No. 4) |

Mouse CB₂ and human CB₂ PCR reactions, yielding respectively a 506 bp and a 464 bp product, were performed using the following conditions: 1 min at 95° C. and 35 cycles (30s at 95° C., 30s at 58° C. and 1 min at 72° C.). Finally, after a final extension step at 72° C. for 5 min, PCR products were separated on 1.5% agarose gels.

### In Vivo Experiments

Adult CB₂ receptor knock-out male mice (8-week old) and their respective wild-type littermates were injected i.p. with 50 mg/kg BrdU daily for 3 days, and perfused 1 day later. HU-308 (15 mg/kg) was administered i.p. for 5 days either alone or in combination with 1 mg/kg SR144528 (injected 30 min before HU-308). Control animals received the corresponding vehicle injection (100 μl PBS supplemented with 0.5 mg defatted bovine serum albumin and 4% dimethylsulfoxide). BrdU was administered daily during the pharmacological administration period. In the case of experiments on kainate-induced excitotoxicity, animals were injected with 15 mg/kg kainate or vehicle. E17.5 mouse embryos from mothers injected twice with 100 mg/kg BrdU (30-min interval between injections) were obtained 1 h after the first injection.

At the end of the study, animals were euthanized and their brains were fixed into 4% paraformaldehyde in PBS until further analysis.

### Immunostaining and Confocal Microscopy

Mice were perfused and immunostaining was performed in 30-μm brain coronal free-floating sections [Rueda, D. et al., J. Biol. Chem. 277, 4645-50, 2002]. Sections were incubated with polyclonal anti-CB₂ antibody together with anti-nestin, anti-Neu, or anti-GFAP antibodies followed by secondary staining for rabbit and mouse IgGs with highly cross-adsorbed AlexaFluor 594 and AlexaFluor 488 secondary antibodies (Molecular Probes), respectively. Neural progenitor proliferation was determined with anti-BrdU antibody and secondary anti-rat IgG-AlexaFluor 594 in sections counterstained with TOTO-3 iodide. Preparations were examined using Leica software and SP2 AOBS microscope with 2 passes with a Kalman filter and a 1024×1024 collection box. BrdU⁺ cells were counted in the subgranular zone and granule cell layer of the dentate gyrus. A 1-in-6 series of adult hippocampal mouse sections located between 1.3 and 2.1 mm posterior to bregma were employed. The number of cells was normalized to the area of the dentate gyrus of each 30-μm section followed by the determination of the total positive cell number per animal. Frozen mouse embryo sections were incubated with anti-BrdU antibody together with Yoyo-1 iodide, and positive cells were determined in 7 sections per animal.

The specificity of CB₂ receptor immunoreactivity was corroborated using CB₂^{-/-} mouse sections, in which no immunoreactivity was observed, and allowed to adjust optimal confocal microscope settings.

Results are expressed as number of BrdU positive cells per section in the dentate gyrus of the animals.

### Statistical Analysis

Results shown represent the means±S.D. of the number of experiments indicated in every case. Statistical analysis was performed by ANOVA. A post hoc analysis was made by the Student-Neuman-Keuls test. In vivo data were analyzed by an unpaired Student t-test.

### Example 1

### Neural Progenitors Express CB₂ Receptors In Vitro and In Vivo

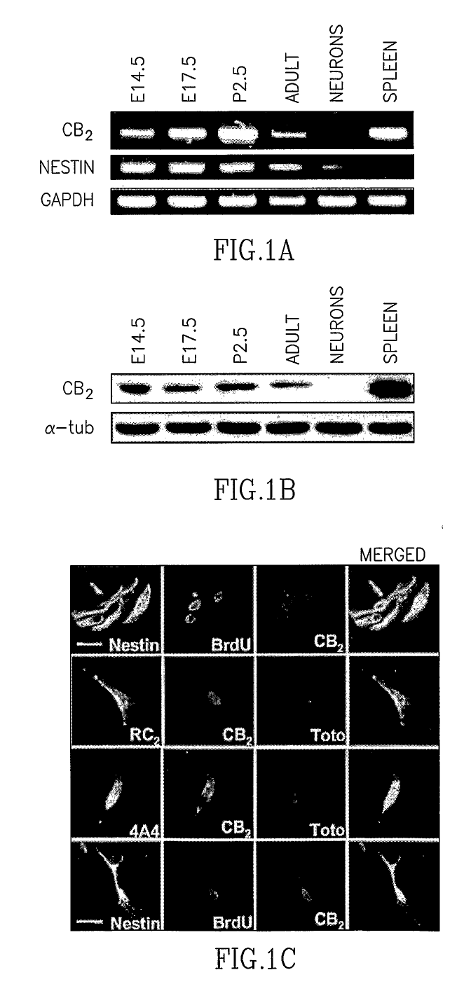

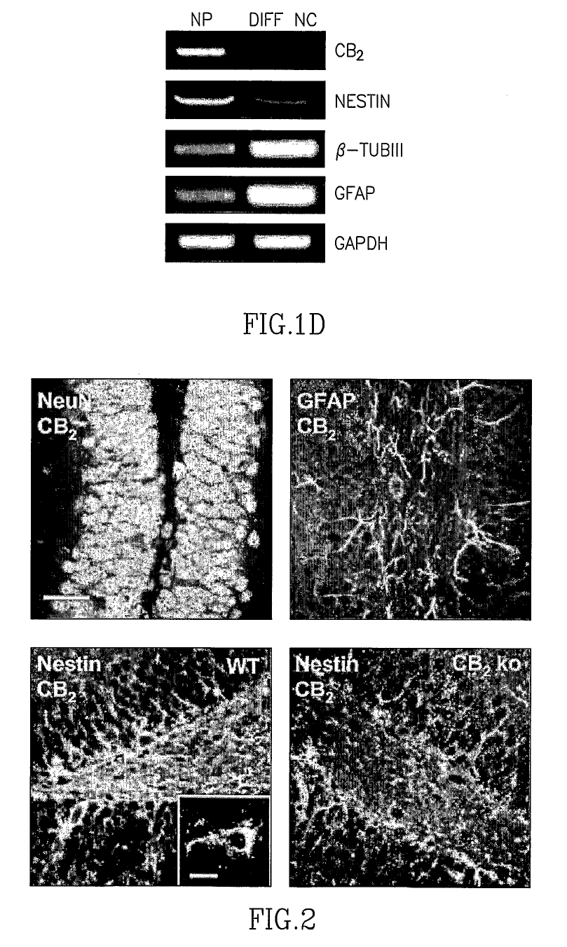

To determine whether neural progenitor cells express CB₂ receptors, clonally-expanded neurospheres derived from embryonic and adult brain were generated. Results are shown in FIG. 1.

FIG. 1 shows that neural progenitors express CB₂ receptors in vitro. In each case GAPDH served as internal house-keeping control in the RT-PCR experiments and α-tubulin served as internal control in the Western blots.

FIG. 1A compares the level of gene expression of the CB₂ receptor and nestin in embryonic (E), postnatal (P) and adult neural progenitors as determined by RT-PCR. Differentiated cortical neurons as well as spleen were used as negative and positive controls, respectively.

FIG. 1B shows the level of protein expression of the CB₂ receptor in the previously mentioned cells and tissues, as determined by Western blot.

FIG. 1C shows the results of a typical immunostaining experiment. Adherent embryonic (four upper slides) and adult (four lower slides) neural progenitor cultures were immunostained with anti-nestin, BrdU and CB₂ receptor antibodies (as indicated). Postnatal radial glial progenitors (middle slides) were labeled against RC2 or phosphorylated-vimentin (green) and the CB₂ receptor (red). Co-localization is shown in the merged images. Scale bars 20 μm.

FIG. 1D shows the analysis of CB₂ receptor expression in undifferentiated neural progenitors (NP) and their differentiated neural cell progeny (Diff NC) evaluated by the presence of nestin, β-tubulin III and GFAP transcripts.

Reverse transcription-PCR (FIG. 1A) and Western blot (FIG. 1B) analyses revealed that neural progenitors express CB₂ receptors and that its presence remains evident as well in adult-derived cells. Next neural progenitors were labeled with antibodies directed against the CB₂ receptor and nestin, a widely used marker of multipotent neuroepithelial cells. As inferred from the co-localization images, it was confirmed that neural progenitor cells, including those actively dividing (as identified by BrdU incorporation), express CB₂ receptors (FIG. 1C, upper panels). Importantly, radial progenitor cells, the postulated continuum lineage from embryonic towards adult neural progenitors, were also positive for CB₂ receptors. Thus, cells expressing the radial glial marker RC2, as well as dividing radial cells identified by an antibody against phosphorylated vimentin, were double-labeled with the anti-CB₂ antibody (FIG. 1C, middle panels). In line with these observations, CB₂ receptor expression persisted in adult neural progenitor cells (FIG. 1C, lower panels). As CB₂ receptor expression is known to be restricted in neural cells, its potential regulation during neural differentiation was investigated next. Thus, neural progenitors were differentiated and CB₂ expression analyzed in parallel with β-tubulin-III and GFAP, markers of neuronal and astroglial cells, respectively. CB₂ receptor expression was abrogated in differentiated cells with the concomitant appearance of the neuronal and astroglial markers (FIG. 1D).

Next confocal microscopy was used to determine whether CB₂ receptors are expressed in vivo in progenitor cells resident in the subgranular zone of the dentate gyrus of the hippocampus, one of the most prominent neurogenic areas throughout lifespan, including adulthood. Results are shown in FIG. 2.

FIG. 2 shows that neural progenitors express CB₂ receptors in vivo. Expression of the CB₂ receptor (red) in neural progenitors (nestin-positive cells; green) but not in mature neurons (NeuN-positive cells; green) and astrocytes (GFAP-positive cells; green) as assessed by confocal microscopy in adult hippocampal sections. Inset shows a high magnification image of a representative double nestin-CB₂ positive cell. Sections from CB₂^{-/-} deficient were employed as specificity controls. Cells were counterstained with TOTO-3 iodide (blue). Scale bars: 40 and 10 μm.

As shown in FIG. 2, CB₂ receptor expression was found only in nestin-positive cells, while its presence could not be detected in differentiated neurons (NeuN-positive cells) and astrocytes (GFAP-positive cells). Altogether, these results show that CB₂ cannabinoid receptors are expressed in neural progenitor cells both during development and in the adulthood and become downregulated with neural cell differentiation.

### Example 2

### CB₂ Receptors Control Neural Progenitor Cell Proliferation In Vitro

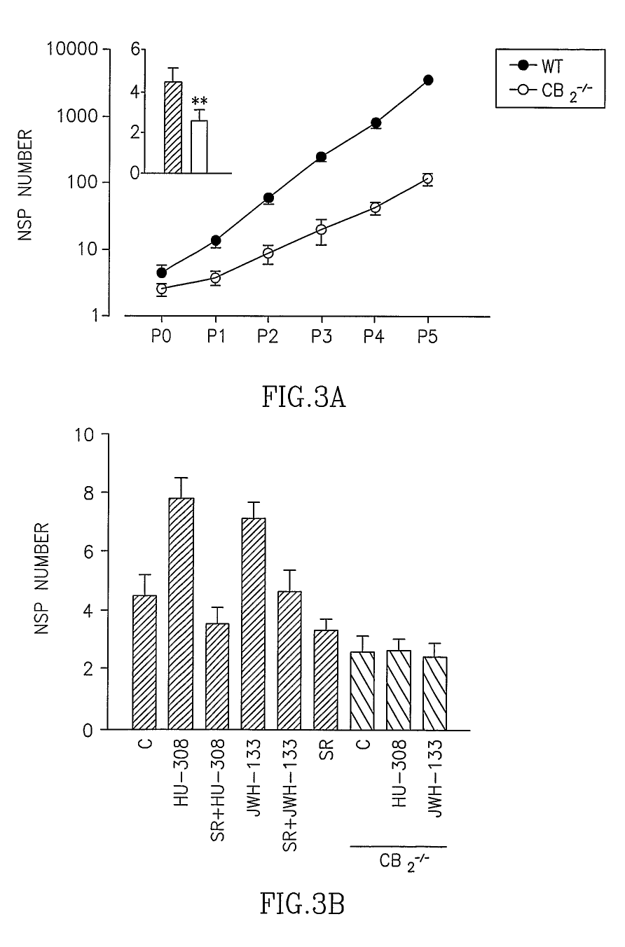

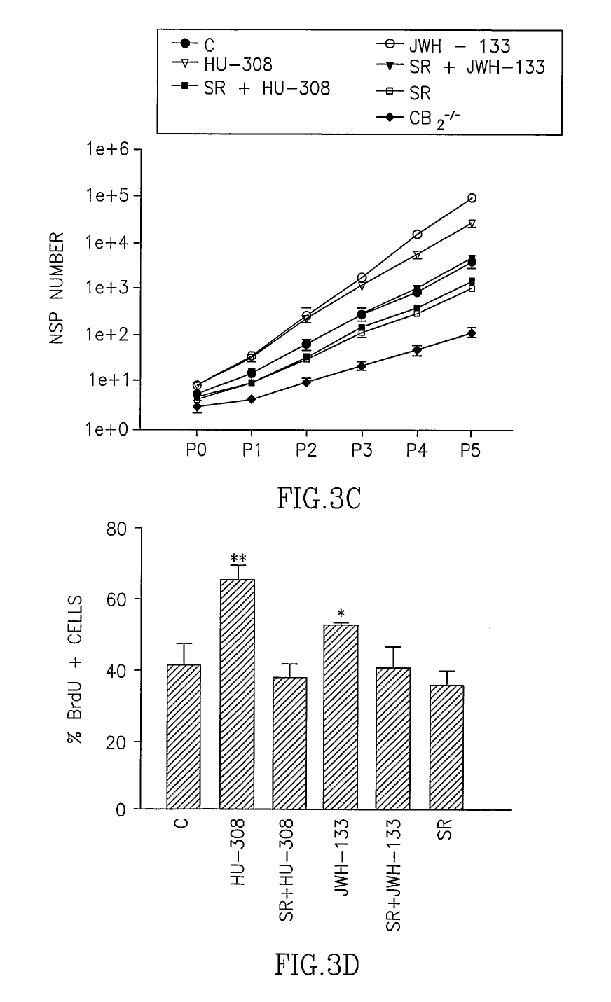

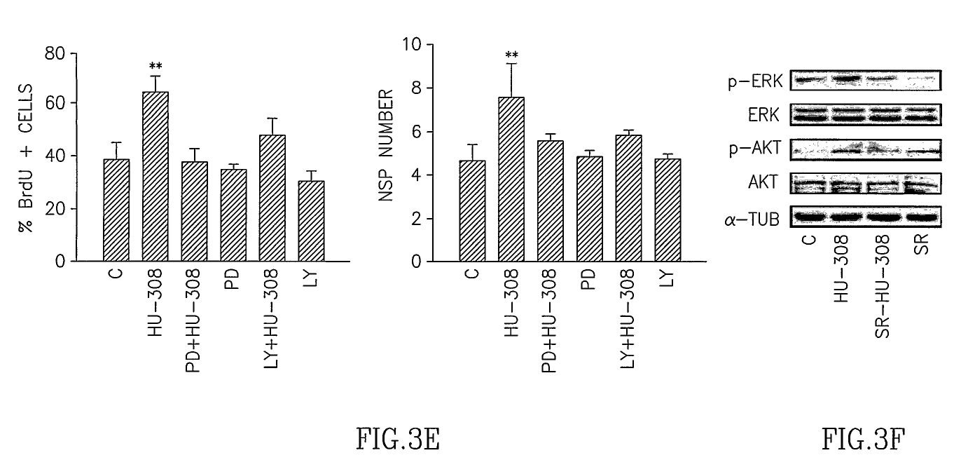

To determine whether CB₂ receptors control neural progenitor cell function, neurospheres from CB₂-deficient mice and their wild-type littermates were first generated. Results are shown in FIG. 3.

FIG. 3 shows that CB₂ receptors control neurosphere generation and neural progenitor cell proliferation in vitro.

FIG. 3A compares the self-renewal ability of E17.5 neural progenitors derived from wild-type (WT) and CB₂−/− mice. The number of neurospheres (NSP) was quantified after 5 consecutive neurosphere passages. Inset: Primary neurosphere generation in the two mouse strains (CB₂^{-/-} white bar).

FIG. 3B depicts the amount of primary neurosphere generated after 7 days of exposure of neural progenitors to vehicle (C), the CB₂-selective agonists HU-308 or JWH-133 (30 nM) and/or the CB₂-selective antagonist SR144528 (2 μM; SR). CB2^{-/-} progenitors were also employed (where indicated).

FIG. 3C shows the self-renewal ability of wild-type neural progenitors incubated with the previously mentioned treatments for 5 consecutive passages, as measured by the amount of neurospheres at each passage. Self-renewal of CB₂-deficient progenitors in the presence of vehicle is also shown (as indicated).

FIG. 3D shows the percentage of BrdU-positive cells from dissociated neurospheres incubated with the previously mentioned treatments for 16 hours.

FIG. 3E shows the percentage of BrdU-positive cells (left panel) and neurosphere generation (right panel) of progenitors treated with vehicle (C), HU-308 (30 nM) and/or PD98059 (10 μM; PD) and/or LY294,002 (5 μM; LY).

FIG. 3F shows ERK and Akt phosphorylation after progenitor challenge with vehicle (C) or HU-308 (alone or in the presence of SR144528) for 15 min (ERK) or 2 min (Akt).

Results correspond to 3 (FIGS. 3A, C, E and F) or 4 (FIGS. 3B and D) independent experiments. Significantly different from control wild-type cells: * P<0.05, ** P<0.01.

Genetic ablation of the CB₂ receptor impaired primary neurosphere generation (FIG. 3A, inset). Moreover, neural progenitor self-renewal, as determined by neurosphere generation for several consecutive passages, was reduced in CB₂-deficient cells (FIG. 3A). The observed impairment of neural progenitor function in CB₂^{-/-} cell cultures prompted us to analyze the prominin (CD-133)-positive subpopulation, as these cells are considered to constitute the stem cell fraction responsible for neurosphere formation activity. Of interest, CB2^{-/-} neurospheres, when compared to wild-type cultures by flow cytometry analysis, showed a reduction in their CD-133⁺ subpopulation (CD-133⁺ cells: 5.8±2.0% versus 7.4±1.5%, respectively).

The functional relevance of the CB₂ receptor was further investigated by incubating neurospheres with selective receptor ligands. Thus, the CB₂-selective agonists HU-308 and JWH-133 increased both primary neurosphere generation (FIG. 3B) and neural progenitor self-renewal (FIG. 3C), and both actions were prevented by the CB₂-selective antagonist SR144528. The selectivity of CB₂ agonists was confirmed by the observation that neither HU-308 nor JWH-133 was able to enhance neurosphere generation in CB₂-deficient neural progenitors (FIG. 3B). Moreover, HU-308 and JWH-133 increased the number of BrdU-incorporating cells in a CB₂-dependent manner (FIG. 3D), supporting the direct impact of CB₂ receptor activation on neural progenitor cell proliferation. Likewise, increased neurosphere generation was observed upon CB₂ receptor activation in postnatal and adult progenitors (percentage of neurosphere number relative to vehicle incubations: HU-308: 130±8% and 161±20%, respectively; JWH-133: 154±22% and 149±6%, respectively), and this action was prevented by SR144528 (data not shown).

In order to determine the potential signaling mechanism responsible for CB₂-mediated proliferation, neural progenitors were incubated in the presence of HU-308 and selective inhibitors of the extracellular signal-regulated kinase (ERK) cascade (PD98059) and the phosphatidylinositol 3-kinase/Akt pathway (LY294,002). HU-308 induction of cell proliferation was prevented by both inhibitors (FIG. 3E, left panel), a finding that was confirmed in neurosphere generation assays (FIG. 3E, right panel). These results prompted us to analyze CB₂-mediated regulation of ERK and Akt. Thus, HU-308 stimulated ERK and Akt, and this action was prevented by SR144528 (FIG. 3F).

### Example 3

### CB₂ Receptors Control Neural Progenitor Cell Proliferation In Vivo

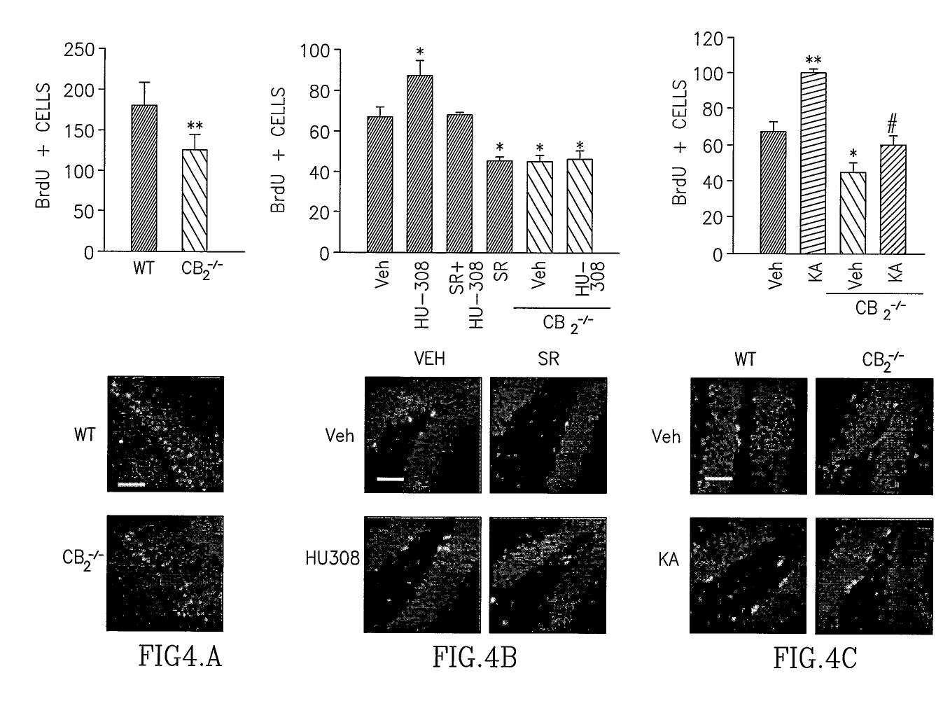

The functional relevance of the CB₂ receptor in controlling neural progenitor cell proliferation in vivo was determined by assessing BrdU incorporation in CB₂-deficient mice and their wild-type littermates. Results are shown in FIG. 4.

FIG. 4 shows that CB₂ receptors control neural progenitor cell proliferation in vivo.

FIG. 4A shows the number of BrdU-positive cells per section in the dentate gyrus of wild-type (WT; n=5) and CB₂^{-/-} (n=7) mouse E17.5 embryos.

FIG. 4B shows the number of BrdU-positive cells per section in the dentate gyrus of wild-type (WT; n=4) and CB₂^{-/-} (n=3) adult mice injected with the indicated agents.

FIG. 4C shows the number of BrdU-positive cells per section in the dentate gyrus of wild-type (WT; n=4) and CB₂^{-/-} (n=4) adult mice injected with saline (Veh) or kainic acid (KA). Lower panels show representative immunostainings of BrdU-positive cells (light) co-stained with TOTO-3 (dark).

Scale bars: 90 μm (A) and 45 μm (B and C). Significantly different from control wild-type mice: * P<0.05, ** P<0.01. Significantly different from knock-out mice treated with kainate: ^{#} P<0.05.

In both embryonic (FIG. 4A) and adult (FIG. 4C) brain, CB₂ knock-out animals showed a significant decrease in BrdU-labeled cells in the dentate gyrus of the hippocampus. These results suggest that neural progenitor proliferation in vivo may be suitable for CB₂ pharmacological manipulation. Thus, HU-308 and/or SR144528 were administered for 5 consecutive days and hippocampal proliferation was determined. Importantly, CB₂ activation increased progenitor proliferation, while CB₂ blockade exerted the opposite action (FIG. 4B). The selectivity of HU-308 in vivo was confirmed by SR144528 antagonism and by the lack of HU-308 agonistic effect in CB₂-deficient mice. The potential role of CB₂ receptors in the control of neural progenitor cell proliferation was further investigated in a situation of brain injury, such as kainate-induced excitotoxicity. As shown in FIG. 4C, the remarkable excitotoxic stimulation of neural progenitor cell proliferation was abrogated in CB₂-deficient mice.

These findings of impaired neural progenitor proliferation after neuroexcitotoxic damage in CB₂-deficient mice, together with the protective role of cannabinoids in a variety of brain damage models, suggest that endocannabinoids generated on demand upon brain injury may enhance neural progenitor proliferation via CB₂ receptors.

The relevance of these results is further strengthened by the recent demonstration of the role of the endocannabinoid system in the regulation of adult neurogenesis. Hippocampal progenitors produce endocannabinoids in a regulated manner and express the CB₁ receptor. In vivo regulation of cannabinoid signaling during central nervous system development alters neuronal activity [Bernard, C. et al., Proc. Natl. Acad. Sci. USA 102, 9388-93, 2005] and generation [Berghuis, P. et al., Proc. Natl. Acad. Sci. USA 102, 19115-20, 2005]. These findings add to the reported impairment of cognitive functions in CB₁ knock-out mice [Bilkei-Gorzo, A. et al., Proc. Natl. Acad. Sci. USA 102, 15670-5, 2005] and the potential of cannabinoid-mediated regulation of adult neurogenesis [Jiang, W. et al., J. Clin. Invest. 115, 3104-16, 2005].

The use of cannabinoids in medicine is severely limited by their well known psychotropic effects. Although psychoactivity tends to disappear with tolerance upon continuous cannabinoid use, it is obvious that cannabinoid-based therapies devoid of side effects would be desirable. As the unwanted effects of cannabinoids are mediated largely or entirely by CB₁ receptors within the brain, the most conceivable possibility would be to use cannabinoids that selectively target CB₂ receptors. In this context, the recent synthesis of CB₂-selective agonists opens an attractive clinical possibility. By showing that CB₂ receptor activation is functional in stimulating neural progenitor cell proliferation in vitro and in vivo, the present report opens the attractive possibility of finding cannabinoid-based therapeutic strategies for neural disorders devoid of non-desired psychotropic effects. Specifically, the proliferative effect of cannabinoids reported here may set the basis for the potential pharmacological modulation of neural progenitor cell fate by CB₂-selective ligands.

To the extent necessary to understand or complete the disclosure of the present invention, all publications, patents, and patent applications mentioned herein are expressly incorporated by reference in their entirety by reference as is fully set forth herein.

Although the present invention has been described with respect to various specific embodiments presented thereof for the sake of illustration only, such specifically disclosed embodiments should not be considered limiting. Many other such embodiments will occur to those skilled in the art based upon applicants' disclosure herein, and applicants propose to be bound only by the spirit and scope of their invention as defined in the appended claims.

1. A method for promoting, inducing and enhancing neurogenesis, comprising administering to an individual in need thereof a prophylactically or therapeutically effective amount of a pharmaceutical composition comprising as an active ingredient a CB₂ selective agonist or an isomer, pharmaceutically acceptable salt, ester, polymorph, solvate or prodrug thereof.

2. The method according to claim 1, wherein the CB₂ selective agonist is selected from the group consisting of an α-pinene derivative, an aminoalkylindole, an anandamide, a 3-aroylindole, an aryl or heteroaryl sulfonate, an arylsulphonamide, a benzamide, a biphenyl-like cannabinoid, a cannabinoid optionally further substituted by one or more fused or bridged mono- or polycyclic rings, a pyrazole-4-carboxamide, an eicosanoid, a dihydroisoindolone, a dihydrooxazole, a quinazolinedione, a quinolinecarboxylic acid amide, a resorcinol derivative, a tetrazine, a triazine, a pyridazine and a pyrimidine derivative, and isomers, pharmaceutically acceptable salts, esters, polymorphs, solvates and prodrugs thereof.

3. The method according to claim 2, wherein the CB₂ selective agonist is a (+)-α-pinene derivative of formula (I): 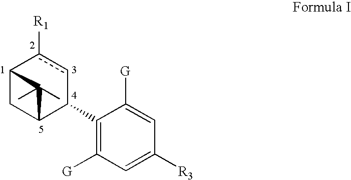
having a specific stereochemistry wherein C-5 is in the (S) configuration, the protons at C-1 and C-5 are cis in relation to one another and the protons at C-4 and C-5 are trans in relation to one another, wherein:
the dashed line between C-2 and C-3 designates an optional double bond;
R₁ is selected from the group consisting of:
(a) —R′ wherein R′ is a C₁-C₅ straight or branched chain alkyl;
(b) —OR″ wherein R″ is a hydrogen or a C₁-C₅ straight or branched chain alkyl optionally containing a terminal —OR′″ or —OC(O)R′″ moiety, wherein R′″ is a hydrogen or a C₁-C₅ straight or branched chain alkyl;
(c) -LN(R″)₂ wherein L is a C₁-C₅ straight or branched chain alkylene and at each occurrence R″ is as previously defined;
(d) -LX wherein L is as previously defined and X is halogen;
(e) -L^{a}C(O)N(R″)₂ wherein L^{a} is a direct bond or a C₁-C₅ straight or branched chain alkylene and R″ is as previously defined;
(f) -L^{a}C(O)OR″ or -L^{a}OC(O)R″ wherein La and R″ are as previously defined; and
(g) -LOR′″ wherein L and R′″ are as previously defined;
G is at each occurrence independently selected from the group consisting of hydrogen, halogen and —OR₂ wherein R₂ is a hydrogen or C₁-C₅ straight or branched chain alkyl optionally containing a terminal —OR′″, —OC(O)R′″, C(O)OR′″, or —C(O)R′″ moiety wherein R′″ is as previously defined; and
R₃ is selected from the group consisting of (a) a C₁-C₁₂ straight or branched chain alkyl; (b) —OR″″ wherein R″″ is a straight or branched chain C₂-C₈ alkyl which can be optionally substituted at the terminal carbon atom by a phenyl group; and (c) —(CH₂)ₙOR′″ wherein n is an integer of 1 to 7 and R′″ is as previously defined;
and pharmaceutically acceptable salts, esters, solvates, polymorphs or prodrugs of said compound.

4. The method according to claim 3, wherein the CB₂ selective agonist is a compound of formula (I) wherein R₁ is CH₂OH, G is OCH₃, R₃ is 1,1-dimethylheptyl and the dashed line between C-2 and C-3 designates a double bond.

5. The method according to claim 1, wherein said pharmaceutical composition further comprises a pharmaceutically acceptable diluent, carrier or excipient.

6. The method according to claim 5, wherein the diluent comprises an aqueous solution comprising a pharmaceutically acceptable cosolvent, a micellar solution prepared with natural or synthetic ionic or non-ionic surfactants, or a combination of such cosolvent and micellar solution.

7. The method according to claim 6, wherein the cosolvent solution comprises a solution of ethanol, a surfactant and water.

8. The method according to claim 5, wherein the carrier is an emulsion comprising a triglyceride, lecithin, an emulsifier, and water.

9. The method according to claim 1, wherein the pharmaceutical composition is in a form suitable for intracerebroventricular, intraparenchymal, intraspinal, intracistemal, intracranial, oral, buccal, mucosal, parenteral, intravenous, intramuscular, intraperitoneal, subcutaneous, transdermal, intrathecal, rectal or intranasal administration.

10. The method according to claim 1, wherein the daily dosage of said CB₂ selective agonist is between 0.01 and 50 mg/kg.

11. The method according to claim 1, wherein the promotion of neurogenesis is used to prevent, alleviate or treat neurological injuries or damages to the CNS or the PNS associated with physical injury, ischemia, neurodegenerative disorders, certain medical procedures or medications, tumors, infections, metabolic or nutritional disorders, cognition or mood disorders, and various medical conditions associated with neural damage or destruction.

12. The method according to claim 11 wherein the physical injury is selected from the group consisting of head trauma, mild to severe traumatic brain injury (TBI), spinal cord injury, diffuse axonal injury, craniocerebral trauma, cranial nerve injuries, cerebral contusion, intracerebral haemorrhage and acute brain swelling.

13. The method according to claim 11 wherein the ischemia results from spinal cord infarction or ischemia, ischemic infarction, stroke, cardiac insufficiency or arrest, atherosclerotic thrombosis, ruptured aneurysm, embolism or haemorrhage.

14. The method according to claim 11 wherein the neurological injury or damage to the CNS or the PNS results from a neurodegenerative disorders selected from the group consisting of Alzheimer's disease (AD), Lewy Body dementia, Parkinson's disease (PD), Huntington's disease (HD), non-Huntingtonian type of Chorea, Pick's disease, Creutzfeldt-Jakob disease (CJD), kuru, Guillain-Barré syndrome, progressive supranuclear palsy; or from neurological lesions associated with diabetic neuropathy, Bell's palsy, systemic lupus erythematosius (SLE), demyelinating disorders, amyotrophic lateral sclerosis (ALS), multiple sclerosis (MS), motor neuron disease, retinal degeneration, muscular dystrophy, Dejerine-Sottas syndrome and peripheral neuropathies.

15. The method according to claim 11 wherein the neurological injury or damage is associated with certain medical procedures, therapies or exposure to biological or chemical toxins or poisons selected from the group consisting of surgery, coronary artery bypass graft (CABG), electroconvulsive therapy, radiation therapy, chemotherapy, anti-neoplastic drugs, immunosuppressive agents, psychoactive, sedative or hypnotic drugs, alcohol, bacterial or industrial toxins, plant poisons, and venomous bites and stings.

16. The method according to claim 11 wherein the tumor is selected from the group consisting of CNS metastasis, intraaxial tumors, primary CNS lymphomas, germ cell tumors, infiltrating and localized gliomas, fibrillary astrocytomas, oligodendrogliomas, ependymomas, pleomorphic xanthoastrocytomas, pilocytic astrocytomas, extraaxial brain tumors, meningiomas, schwannomas, neurofibromas, pituitary tumors, and mesenchymal tumors of the skull, spine and dura matter.

17. The method according to claim 11 wherein the infection of bacterial, viral, fungal, parasitic or other origin is selected from the group consisting of pyrogenic infections, meningitis, tuberculosis, syphilis, encephalomyelitis and leptomeningitis.

18. The method according to claim 11 wherein the neurological injury or damage to the CNS or the PNS is associated with metabolic or nutritional disorders selected from the group consisting of glycogen storage diseases, acid lipase diseases, Wemicke's or Marchiafava-Bignami's disease, Lesch-Nyhan syndrome, Farber's disease, gangliosidoses, vitamin B12 and folic acid deficiency.

19. The method according to claim 11 wherein the neurological injury or damage to the CNS or the PNS associated with cognition or mood disorders is selected from the group consisting of learning or memory disorder, bipolar disorders and depression.

20. The method according to claim 11 wherein the medical condition associated with neural damage or destruction is selected from the group consisting of asphyxia, prematurity in infants, perinatal distress, gaseous intoxication for instance from carbon monoxide or ammonia, coma, hypoglycaemia, dementia, epilepsy and hypertensive crises.

21. A method for promoting, inducing and enhancing neurogenesis in vitro, comprising:
a) harvesting neural stem cells and/or progenitors cells from an autologous or heterologous donor;
b) culturing said cells in presence of an effective amount of a CB₂ selective agonist or an isomer, pharmaceutically acceptable salt, ester, polymorph, solvate or prodrug thereof;
c) monitoring the differentiation until desired maturity is reached; and
d) harvesting the partly or fully differentiated neural cells.

22. The method according to claim 21 further comprising transplanting an effective amount of the differentiated cells into the neural tissue of an individual in need thereof in order to prevent, alleviate or treat neurological injuries or damages to the CNS or the PNS associated with physical injury, ischemia, neurodegenerative disorders, certain medical procedures or medications, tumors, infections, metabolic or nutritional disorders, cognition or mood disorders, and various medical conditions associated with neural damage or destruction.


SEQUENCE LISTING (4 sequences)

SEQ ID NO: 1 - DNA, 22 bp, Artificial
  Single strand DNA oligonucleotide
ggatgccgggagacagaagtga

SEQ ID NO: 2 - DNA, 23 bp, Artificial
  Single strand DNA oligonucleotide
cccatgagcggcaggtaagaaat

SEQ ID NO: 3 - DNA, 23 bp, Artificial
  Single strand DNA oligonucleotide
caacccaaagccttctagacaag

SEQ ID NO: 4 - DNA, 21 bp, Artificial
  Single strand DNA oligonucleotide
gtggatagcgcaggcagaggt